SEQUENCE LISTING (8 sequences)

SEQ ID NO: 1 - DNA, 18 bp, Artificial Sequence
  Synthetic primer
cctgcactttatctatcg

SEQ ID NO: 2 - DNA, 24 bp, Artificial Sequence
  Synthetic primer
tcaaagaacaagtttgataactaa

SEQ ID NO: 3 - DNA, 26 bp, Artificial Sequence
  Synthetic primer
tttgagaggtttactcttaaaacatg

SEQ ID NO: 4 - DNA, 19 bp, Artificial Sequence
  Synthetic primer
ccaaattaaaacatatcgt

SEQ ID NO: 5 - DNA, 18 bp, Artificial Sequence
  Synthetic primer
tgagtacgttatttaagg

SEQ ID NO: 6 - DNA, 18 bp, Artificial Sequence
  Synthetic primer
agcagatacaatgcgatc

SEQ ID NO: 7 - DNA, 21 bp, Artificial Sequence
  Synthetic primer
attgtagtttacgcctatgta

SEQ ID NO: 8 - DNA, 23 bp, Artificial Sequence
  Synthetic primer
acgtagctatgtattttacagag

(19) United States
(12) Patent Application Publication
(10) Pub. No.: US 2014/0199697 A1
(43) Pub. Date: Jul. 17, 2014

(54) QUANTIFICATION AND MOLECULAR DETECTION OF LACTIC ACID BACTERIA IN A SAMPLE

(71) Applicants: Mindy M. Brashears, Wolfforth, TX (US); Kendra Nightingale, Lubbock, TX (US); Guy Loneragan, Lubbock, TX (US); Qingli Zhang, Lubbock, TX (US)

(72) Inventors: Mindy M. Brashears, Wolfforth, TX (US); Kendra Nightingale, Lubbock, TX (US); Guy Loneragan, Lubbock, TX (US); Qingli Zhang, Lubbock, TX (US)

(73) Assignee: TEXAS TECH UNIVERSITY SYSTEM, Lubbock, TX (US)

(21) Appl. No.: 14/154,824

(22) Filed: Jan. 14, 2014

Related U.S. Application Data
(60) Provisional application No. 61/753,191, filed on Jan. 16, 2013.

(51) Int. Cl. C12Q 1/68; C12Q 1/06

(52) U.S. Cl. CPC C12Q 1/689; C12Q 1/06
USPC 435/6.11; 435/39; 435/6.12; 435/6.15; 536/24.32; 536/24.33

(57) ABSTRACT

Methods and compositions are disclosed for detection of probiotic bacteria strains intentionally provided to animals comprising: providing an animal with a known amount or number of probiotic bacteria; following a pre-determined time, obtaining a biological sample suspected of comprising the inoculated probiotic bacteria from the animal; and quantitatively detecting the amount of probiotic bacteria in the biological sample.

### CROSS-REFERENCE TO RELATED APPLICATIONS

This application claims priority to U.S. Provisional Application Ser. No. 61/753,191, filed Jan. 16, 2013, the entire contents of which are incorporated herein by reference.

### TECHNICAL FIELD OF THE INVENTION

The present invention relates in general to the field of detection and quantification of lactic acid producing bacteria in a sample, and more particularly, to detection and quantification of both lactic acid utilizing bacteria and lactic acid producing bacteria in a sample.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jan. 14, 2014, is named TECH1110US_SeqList.txt and is 2, kilobytes in size.

### BACKGROUND OF THE INVENTION

Without limiting the scope of the invention, its background is described in connection with methods for improved feed efficiency in animals.

Improving feed efficiency and animal health has been the primary objectives in the animal industry. As the prices of feed and fuel increase, achieving higher feed efficiency is becoming even more important. Lactic acid producing bacteria and lactic acid utilizing bacteria have been shown to improve feed efficiency in ruminants and poultry when used as a feed supplement. See, e.g., U.S. Pat. No. 5,534,271. These bacteria have also been shown to reduce pathogenic infection in animals.

### SUMMARY OF THE INVENTION

The present invention includes a method for detection of probiotic bacteria strains intentionally provided to animals comprising: providing an animal with a known amount or number of probiotic bacteria; following a pre-determined time, obtaining a biological sample suspected of comprising the inoculated probiotic bacteria from the animal; and quantitatively detecting the amount of probiotic bacteria in the biological sample. In one aspect, the method further comprises the step of modifying the amount of probiotic bacteria inoculated into a feed based on the amount wherein the probiotic bacteria detected in the biological sample. In another aspect, the step of quantitatively detecting the amount of probiotic bacteria in the biological sample is by a nucleic acid amplification assay, a nucleic acid detection assay, an oligonucleotide ligation assay (OLA), a primer probe assay, a polymerase chain reaction (PCR), a quantitative polymerase chain reaction (qPCR), multiplex-PCR, multiplex qPCR, a reverse-transcriptase polymerase chain reaction (RT-PCR), a ligase chain reaction (LCR), a polynomial amplification method, DNA sequencing method, or an method comprising primer extension. In another aspect, the step of quantitatively detecting the amount of probiotic bacteria in the biological sample is by qPCR it is selected from at least one of competitive, noncompetitive, or kinetic. In another aspect, the probiotic is a combination of a lactic acid producing bacterium and a lactic acid utilizing bacterium. In another aspect, the pre-determined amount of probiotic bacterium is selected from at least one of 1×10, 1×10³, 1×10⁴, 1×10⁵, 1×10⁶, 1×10⁷, 1×10⁸, 1×10⁹, or 1×10¹⁰ cfu per gram of a feed.

In another embodiment, the method includes quantifying a probiotic bacteria in a sample, said method comprising: providing an animal with a feed inoculant comprising a known amount or number of probiotic bacteria; following a pre-determined time, obtaining a biological sample suspected of comprising the inoculated probiotic bacteria from the animal; generating a DNA fragment by amplifying a segment of genetic material of the probiotic bacterium, and quantifying the amount of the probiotic bacterium, wherein the amplification using a pair of oligonucleotide primers, wherein the pair of oligonucleotides has at least 90% sequence identity with oligonucleotides of SEQ ID. No. 1 and SEQ ID. No. 2, respectively. In another aspect, the method further comprises a step of culturing the probiotic bacterium in a medium and enumerating colonies formed by the lactic acid producing bacterium. In another aspect, the probiotic bacteria is selected from at least one of LA51 strain or PF24. In another aspect, the probiotic bacteria is mixed with animal feed. In another aspect, the biological sample is a fecal sample, a biopsy, a saliva sample, or a lymph node sample. In another aspect, the probiotic bacterium is selected from at least one of Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, Megasphaera elsdenii, Peptostreptococcus asaccharolyticus, Propionibacterium freudenreichii, Propionibacterium acidipropionici, Propionibacterium globosum, Propionibacterium jensenii, Propionibacterium shermanii, Propionibacterium spp., or Selenomonas ruminantium. In another aspect, the probiotic bacteria are a lactic acid producing bacterium is the LA51 strain, and the lactic acid utilizing bacterium is the PF24 strain.

The present disclosure advances the art by providing methods and compositions for detecting and/or quantifying the amount of a lactic acid bacterium in a sample. In one embodiment, the sample may be a feed sample, a fecal sample, or a sample taken from inside the digestive system of an animal. In another embodiment, the disclosed methods may allow for direct plating to detect both NP51 (LA51) and PF24 in the same sample.

The disclosed composition may contain one or more primers which may be used to generate a DNA fragment by amplifying a segment of genetic material from a lactic acid producing bacterium. In one embodiment, the composition may contain an oligonucleotide, wherein the oligonucleotide may have at least 90%, 95%, 99% or 100% sequence identity with SEQ ID. No. 1 or SEQ ID. No. 2. In another embodiment, the composition may contain a pair of oligonucleotides, wherein the pair of oligonucleotides may have at least 90%, 95%, 99% or 100% sequence identity with SEQ ID. No. 1 and SEQ ID. No. 2, respectively. The genetic material may be prepared by extracting total DNA from the lactic acid producing bacterium in the sample. The lactic acid producing bacterium of interest may be mixed together in the sample along with many other microorganisms. In one aspect, the disclosed primers specifically anneal to the genetic material of the subject lactic acid producing bacterium such that only the DNA of the subject lactic acid producing bacterium is amplified. The amplified DNA fragment may be separated by electrophoresis and may be detected and quantified by various methods. The detection methods may include but are not limited to staining with DNA dye, fluorescence labeling, or radioactive labeling. In another aspect, the amplification process is polymerase chain reaction (PCR). In another aspect, the primer may be pre-labeled before the PCR reaction so that the amplified product is labeled when synthesized.

The amount of the DNA fragment obtained in step (a) may be quantified in order to determine the amount of the lactic acid producing bacterium in the sample. In one embodiment, a standard curve may be built by plotting the amount the Cycle Threshold (Ct) against the initial quantities of DNA in the sample. This standard curve may be used to determine the initial quantities of DNA in the sample. Ct is a value measuring the number of cycles that is required for a specific PCR reaction to reach a threshold level of fluorescence signal. In one aspect, the quantity of the DNA from the bacteria of interest in the unknown sample can be determined based on the standard curve, the cycle threshold (Ct) of standard and the unknown samples and the R square (RSq). In one aspect, the negative samples have a Ct of 0. In another aspect, the R square values are between 0.95 and 0.99.

The colony forming unit (CFU) of the lactic acid producing bacterium in the sample may be measured by plating a serial dilution of the lactic acid producing bacterium in a solid medium and by counting the number of colonies after a period of incubation to allow the bacteria to grow and form colonies. In another embodiment, a standard curve may be established by plotting the amount of amplified DNA against the colony forming unit (CFU) of the lactic acid producing bacterium in the sample. This standard curve, along with the amount of amplified DNA obtained from each sample may be used to determine the number of viable bacteria in the sample. In another embodiment, the plating results may be used to confirm and verify the PCR results. See, e.g., Table 1.

Various parameters may determine whether or not the amount of the amplified DNA is proportional to the number of lactic acid producing bacterium in the sample. Such parameters may include but are not limited to duration of each cycle and number of cycles during the PCR reaction. These parameters may be adjusted such that the amount of the amplified DNA is proportional to the number of lactic acid producing bacteria in the sample.

In another embodiment, the sample may contain a lactic acid utilizing bacterium. Similar to the methodology described for quantifying lactic acid producing bacteria, primers may be designed for the lactic acid utilizing bacterium and DNA amplification may be performed using DNA isolated from the lactic acid utilizing bacterium as a template.

In another embodiment, the sample may contain one or more different bacteria. For instance, the sample may contain a mixture of lactic acid producing bacteria and lactic acid utilizing bacteria. Similar methodology may be applied to quantify the different bacteria.

Examples of lactic acid producing bacteria may include but are not limited to the genus of Lactobacillus. More particularly, at least one of the lactic acid producing bacteria may be Lactobacillus acidophilus. Examples of Lactobacillus strains may include but are not limited to LA51 (also known as NP51), M35, LA45, NP28 (also known as C28) and L411 strains.

Examples of lactic acid utilizing bacteria may include but are not limited to the genus of Propionibacterium. More particularly, at least one of the lactic acid producing bacteria may be Propionibacterium freudenreichii. Examples of Propionibacterium strains may include but are not limited to P9, PF24, P42, P93 and P99 strains.

In another embodiment, a method is disclosed for enumerating a lactic acid producing bacterium and a lactic acid utilizing bacterium in the same sample. A portion of the sample may be plated on a first medium to allow both the lactic acid producing bacterium and the lactic acid utilizing bacterium to grow on the first medium. Another portion of the sample may be plated on a second medium to allow only the lactic acid producing bacterium or only the lactic acid utilizing bacterium to grow in the second medium. The number of colonies on the first and second media may be counted to calculate the number of viable lactic acid producing bacteria and viable lactic acid utilizing bacteria in the original sample. This culture method may be combined with the PCR method described herein to quantify the lactic acid bacteria in the sample.

### BRIEF DESCRIPTION OF THE DRAWINGS

For a more complete understanding of the features and advantages of the present invention, reference is now made to the detailed description of the invention along with the accompanying figures and in which:

FIG. 1 shows a standard curve established by plotting Cycle Threshold (Ct) against the initial quantities of DNA in the sample.

FIG. 2 shows a real time PCR amplification plot showing the relationship between the amount of amplified PCR products and the number of cycles performed in the PCR.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

It is to be noted that, as used in this specification and the claims, the singular forms “a,” “an,” and “the” include plural referents unless the context clearly dictates otherwise. Thus, for example, reference to “a pathogen” includes reference to a mixture of two or more pathogens, reference to “a lactic acid producing bacterium” includes reference to one or more lactic acid producing bacteria.

The present inventors provide herein methods and compositions for quantifying bacteria in a sample. More particularly, the disclosure provides primers and methods for quantifying lactic acid producing bacteria or lactic acid utilizing bacteria in a sample, such as a feed sample. For purpose of this disclosure, the term “lactic acid bacteria” may be used to lactic acid producing bacteria (LAB) and lactic acid utilizing bacteria collectively. It is important to quantifying bacteria because it has been found that the dosage of lactic acid bacteria may have certain effects on the effectiveness of the supplement. Underdosage of the bacterial supplement may lessen the effectiveness of the supplement. On the other hand, overdosage may raise the cost of the supplement without additional benefits. Thus, it is desirable to quantify the lactic acid bacteria in a feed sample. However, no effective methods for quantifying lactic acid bacteria have been reported. Traditional culturing methods may be take days or weeks to complete and require relatively large amount of a sample. Although PCR technique has been around for decades, no effective methods for detecting and quantifying lactic acid bacterial strains (including but not limited to NP51 and PF24) based on quantitative polymerase chain reaction (PCR) have been reported.

As used herein, the term “pre-determined time” refers to an amount of time in which the bacteria provided to the animal in the form of an inoculation, a feed that has been inoculated with or has grown in the presence of the known probiotic bacteria, in a liquid form, ingested or in any way delivered to the gastrointestinal tract of the animal, will generally be enough to deliver a sufficient amount of bacteria to the animal such that it can be isolated from a site other than where it was inoculated or provided to the animal. For example, the pre-determined time, could be 1, 2, 3, 4, 5, 10, 15, 20, 25, 30, 35, 40, 45, 50, 55, or 60 minutes. The pre-determined time, could also be 1, 2, 3, 4, 5, 6, 7, 8, 9, 10, 11, 12, 13, 14, 15, 20, 24, 25, 30, 36, 42, 48, 60, 72 or more hours. The predetermined tine could also be 1, 2, 3, 4, 5, 6, 7, 10, 14, 18, 21, 28, or 30 days.

In one embodiment, the lactic acid producing bacterium may include one or more of the following: Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, and combinations thereof.

Examples of lactate utilizing bacterium may include Megasphaera elsdenii, Peptostreptococcus asaccharolyticus, Propionibacterium freudenreichii, Propionibacterium acidipropionici, Propionibacterium globosum, Propionibacterium jensenii, Propionibacterium shermanii, Propionibacterium spp., Selenomonas ruminantium, and combinations thereof.

In one embodiment, the lactic acid producing bacterium is Lactobacillus acidophilus or Lactobacillus animalis. Examples of the lactic acid producing bacterium strains may include but are not limited to the LA51, M35, LA45, NP28, and L411. In another embodiment, the lactic acid producing bacterium strain is LA51. The term Lactobacillus acidophilus/animalis may be used to indicate that either Lactobacillus acidophilus or Lactobacillus animalis may be used. It is worth noting that when strain LA51 was first isolated, it was identified as a Lactobacillus acidophilus by using an identification method based on positive or negative reactions to an array of growth substrates and other compounds (e.g., API 50-CHL or Biolog test). Using modern genetic methods, however, strain LA51 has recently been identified as belonging to the species Lactobacillus animalis (unpublished results). Regardless of the possible taxonomic changes for LA51, the strain LA51 remains the same as the one that has been deposited with ATCC.

Lactobacillus strains C28, M35, LA45 and LA51 strains were deposited with the American Type Culture Collection (ATCC) on May 25, 2005 and have the Deposit numbers of PTA-6748, PTA-6751, PTA-6749 and PTA-6750, respectively. Lactobacillus strain L411 was deposited with the American Type Culture Collection (ATCC) on Jun. 30, 2005 and has the Deposit number PTA-6820. Examples of Propionibacterium freudenreichii strains may include but are not limited to the P9, PF24, P42, P93 and P99 strains. Propionibacterium strain PF24 was deposited with the ATCC on May 25, 2005 and has the Deposit numbers of PTA-6752. P9 and P42 were deposited with the ATCC on Jun. 30, 2005 and have the Deposit numbers of PTA-6821 and PTA-6822, respectively.

The following examples are provided to illustrate the present disclosure, but are not intended to be limiting. The feed ingredients and supplements are presented as typical components, and various substitutions or modifications may be made in view of the foregoing disclosure by one of skills in the art without departing from the principle and spirit of the present invention.

Various commercially available products are described or used in this disclosure. It is to be recognized that these products or associated trade names are cited for purpose of illustration only. Certain physical or chemical properties and ingredients of the products may be modified without departing from the spirit of the present disclosure. One of ordinary skill in the art may appreciate that under certain circumstances, it may be more desirable or more convenient to alter the physical and/or chemical characteristics or composition of one or more of these products in order to achieve the same or similar objectives as taught by this disclosure. It is to be recognized that certain products or organisms may be marketed under different trade names which may in fact be identical to the products or organisms described herein.

The PCR and the plating assays described in the Examples contain ingredients that are in sizes suitable for a small scale setting. It is important to note that these small scale tests may be scaled up for larger sample size. The principle of operation and the proportion of each ingredient in the system disclosed herein may equally apply to a larger scale feed sample test system. Unless otherwise specified, the percentages of ingredients used in this disclosure are on a w/w basis.

### Example 1

### Enumeration of Lactic Acid Bacteria in Feed Samples

This example describes the enumeration of lactic acid producing bacteria (LAB) and/or lactic acid utilizing bacteria (referred to collectively as “lactic acid bacteria” in this disclosure) in a feed sample. Lactobacillus strain NP51 and Propionibacterium strain PF24 were used as examples to illustrate the inventions.

NP51 Enumeration: On Day One, 10 grams of feed sample were weighed and 90 ml of commercially available buffered peptone water (BPW) (containing 1.0 g of peptone and 8.5 g of sodium chloride/liter) (with 0.1% Tween 80) was added to the feed sample to enrich the sample. The enriched sample was stomached for 3 minutes at 230 rpm and the supernatant was collected. The supernatant was then subject to serial dilutions in 9 ml of BPW. The serial-diluted supernatants (10⁻², 10⁻³ and 10⁻⁴ diluted) were plated on a number of plates containing MRS agar (Difco). The MRS agar contained 1.0% peptone, 0.8% meat extract, 0.4% yeast extract, 2.0% glucose, 0.5% sodium acetate trihydrate, 0.1% polysorbate 80 (also known as Tween 80), 0.2% dipotassium hydrogen phosphate, 0.2% triammonium citrate, 0.02% magnesium sulfate heptahydrate, 0.005% manganese sulfate tetrahydrate, 1.0% agar with pH adjusted to 6.2 at 25° C. The MRS plates were incubated at 37° C. for 48 hours. On Day Three, viable colonies of NP51 on the plates were counted. The number of these colonies may be used to calculate the number of viable cells (or colony formation unit (CFU)) in the original sample.

NP51 and PF24 Enumeration: On Day One, 10 grams of feed sample were weighed and 90 ml of BPW (described above) with 0.1% Tween was added to the feed sample to enrich the sample. The enriched sample was stomached for 3 minutes at 230 rpm and the supernatant was collected. The supernatant was then subject to serial dilutions in 9 ml of BPW having 0.1% Tween. The serial-diluted supernatants (10⁻², 10⁻³ and 10⁻⁴ diluted) were plated on a group of plates containing MRS agar (described above) and a second group of plates containing Sodium Lactate (NaLa) agar, which contained 10 g trypticase, 10 g yeast extract, 10 g sodium lactate, 0.25 g disodium phosphate, 15 g of agar and distilled water in 1 liter. The MRS plates were incubated at 37° C. for 48 hours, while the NaLa agar plates were anaerobically incubated at 30° C. for 72 hours. On Day Three, viable counts of NP51+PF24 on MRS agar were enumerated. On Day Four, viable counts of PF24 on NaLa agar were enumerated. The viable number of NP51 may be calculated based on these data.

### Example 2

### NP51 Quantitative PCR (qPCR) Assay

This Example describes the quantitative real time PCR (qRTi-PCR) assay to determine the amount of a specific lactic acid bacterial strain by using primers and probes that are specific to that strain.

90 ml of BPW (containing 0.1% Tween 80) and 10 grams of feed sample were mixed in a stomacher bag and the mixture was stomached at 230 rpm for 3 minutes. DNA from the feed sample was extracted by following the extraction protocol from Mobio PowerFood™ Microbial DNA Isolation.

The following specific primers were used for PCR amplification of a unique DNA fragment from the total DNA extract of NP51 strain: Forward primer: NP51 prev1F: 5′-CCTGCACTTTATCTATCG-3′ (SEQ ID NO. 1); Reverse Primer: NP51 R: 5′-TCAAAGAACAAGTTTGATAACTAA-3′ (SEQ ID NO. 2). The sequence of the probe for detection of the amplified product is as follows: 5′-6-FAM/TTTGAGAGGTTTACTCTTAAAACATG/3′-BHQ1 (SEQ ID NO. 3).

In one embodiment, a pair of primers was used as internal positive control. Their sequences are listed below: F-5′-CCAAATTAAAACATATCGT-3′ (SEQ ID NO. 4); and R-5′-TGAGTACGTTATTTAAGG-3′ (SEQ ID NO. 5).

In another embodiment, another pair of primers was used for amplicons in the positive internal control: F-5′-AGCAGATACAATGCGATC-3′ (SEQ ID NO. 6); and R-5′-ATTGTAGTTTACGCCTATGTA-3′ (SEQ ID NO. 7). The probe is 5′-Hex-ACGTAGCTATGTATTTTACAGAG-3′-BHQ-1 (SEQ ID NO. 8). The primers were resuspended TE to 100 uM and kept at −20° C. until use. The working solutions for the primers and probes were 10 uM for the primers and 25 uM for the probes.

The following supplies were obtained from Agilent Technologies (www.agilent.com): Optical Caps-cat #401425, QPCR 96 well plates (semi-skirted)-cat#401334, and Brilliant II QPCR Low ROX Master Mix, 1 Pack-cat#600806.

A Master Mix (Agilent Mix) containing primers, probe and water was performed under sterile conditions in the PCR room to avoid DNA contamination. The Master Mix contained the following: 12.5 ul of Brillinat II Master mix, 2.25 ul of NP51 Forward Primer (10 uM), 2.25 ul of NP51 Forward Primer (10 uM), 0.25 ul of NP51 bhq1 probe (25 uM), and 5.25 ul of NF H20. Once the Master Mix was performed, 22.5 ul of the Master Mix was distributed into each well of a 96-well PCR plate. 2.5 ul of DNA from each sample to be analyzed is added into the corresponding well. Pipetting was performed very carefully to add exactly 2.5 ul in each well. Unknown and standard samples were analyzed in duplicates.

The 96-well PCR plate was placed into the machine to perform the analysis. The cycling conditions were: 10 minutes of polymerase activation at 95° C., followed by 35 cycles with each cycle being 95° C. for 15 seconds and 60° C. for 60 seconds.

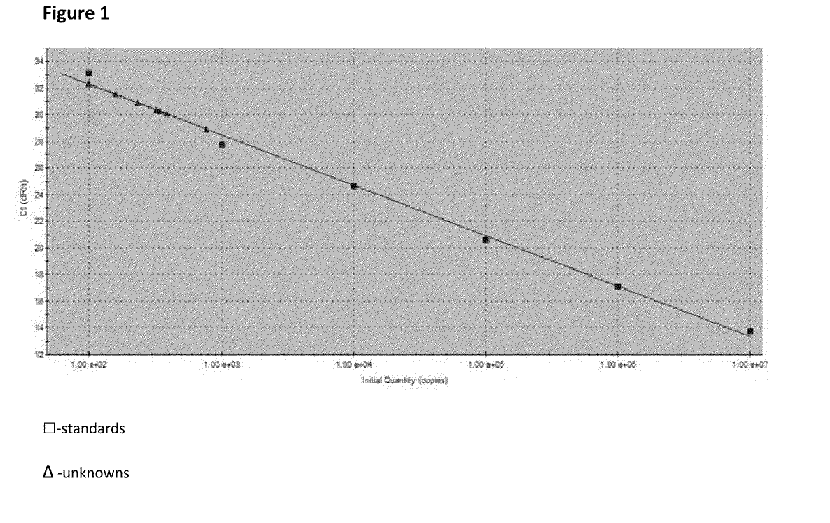

FIG. 1 shows a standard curve established by plotting Cycle Threshold (Ct) against the initial quantities of DNA in the sample. Ct is a value measuring the number of cycles that is required for a specific PCR reaction to reach a threshold level of fluorescence signal. This standard curve may be used to determine the initial quantities of DNA in the sample. Negative samples should have a Ct of 0, while the R square values were between 0.95 and 0.99.

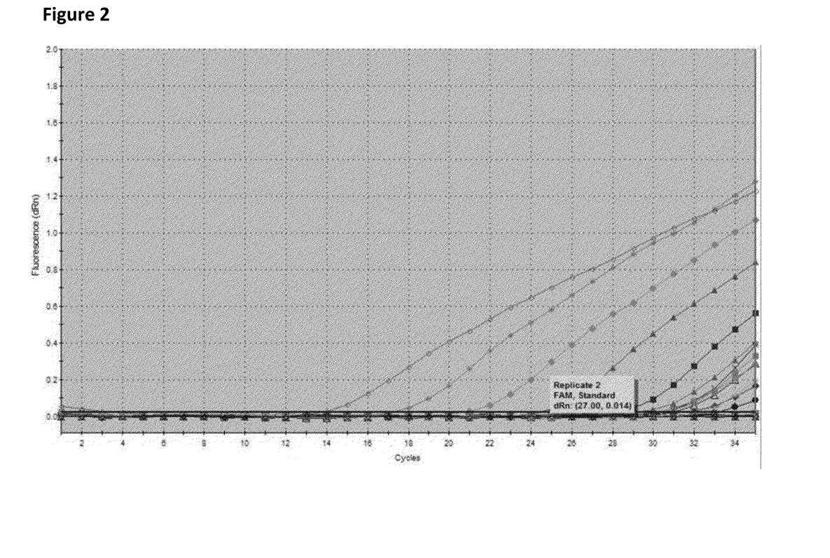

FIG. 2 is a real time PCR amplification plot showing the relationship between the amount of amplified PCR products and the number of cycles performed in the PCR. The linear portion of each curve is the exponential phase of the PCR. The threshold fluorescence is set in the linear portion of the curve.

### Example 3

### Comparison of Microbiological and qPCR Results

Plating and CFU counting may be used to confirm and verify the quantitative PCR results. As shown in Table 1, the results from qPCR and the results from microbiological culturing are in agreement with each other.

TABLE 1
COMPARISON OF MICROBIOLOGICAL AND PCR RESULTS

| | | NP51 | Quantity | Quantity | |
|---|---|---|---|---|---|
| Lactobacilli (CFU/g) | | (Ct) | (copies)/2.5 ul | (copies)/100 ul | RSq |
| Control 1 | 3.1 × 10\hat{ }4 | No Ct | No Ct | No Ct | 0.997 |
| Control 2 | 6.0 × 10\hat{ }4 | 37.77 | No Ct | No Ct | |
| Bov 1 | 1.7 × 10\hat{ }5 | 34.3 | 3.6 × 10\hat{ }3 | 1.4 × 10\hat{ }5 | |
| Bov 2 | 9.5 × 10\hat{ }4 | 31.4 | 4.3 × 10\hat{ }3 | 1.7 × 10\hat{ }5 | |
| Control 1 | 6.0 × 10\hat{ }5 | No Ct | No Ct | No Ct | 0.996 |
| Control 2 | 5.8 × 10\hat{ }5 | No Ct | No Ct | No Ct | |
| Bov 1 | 7.6 × 10\hat{ }5 | 38.89 | 2.89 × 10\hat{ }2 | 1.2 × 10\hat{ }4 | |
| Bov 2 | 8.0 × 10\hat{ }5 | 34.67 | 2.13 × 10\hat{ }2 | 8.5 × 10\hat{ }3 | |
| Control 1 | 2.4 × 10\hat{ }4 | 36.7 | 2.55 × 10\hat{ }1 | 1.0 × 10\hat{ }3 | 0.995 |
| Control 2 | 4.7 × 10\hat{ }4 | 34.99 | 9.85 × 10\hat{ }2 | 4.0 × 10\hat{ }4 | |
| Bov 1 | 1.4 × 10\hat{ }6 | 32.27 | 5.39 × 10\hat{ }2 | 2.2 × 10\hat{ }4 | |
| Bov 2 | 8.5 × 10\hat{ }5 | 30.88 | 1.25 × 10\hat{ }3 | 5.0 × 10\hat{ }4 | |
| Control 1 | 1.6 × 10\hat{ }5 | 28.59 | 1.94 × 10\hat{ }3 | 7.7 × 10\hat{ }4 | 0.949 |
| Control 2 | 1.0 × 10\hat{ }5 | 26.99 | 6.36 × 10\hat{ }3 | 2.5 × 10\hat{ }5 | |
| Bov 1 | 7.0 × 10\hat{ }4 | 29.41 | 1.06 × 10\hat{ }3 | 4.2 × 10\hat{ }4 | |
| Bov 2 | 7.1 × 10\hat{ }4 | No Ct | No Ct | No Ct | |
| Control 1 | 7.5 × 10\hat{ }3 | No Ct | No Ct | No Ct | 0.994 |
| Control 2 | 7.0 × 10\hat{ }3 | No Ct | No Ct | No Ct | |
| Bov 1 | 1.7 × 10\hat{ }4 | 30.36 | 3.24 × 10\hat{ }2 | 1.3 × 10\hat{ }4 | |
| Bov 2 | 1.4 × 10\hat{ }4 | 30.9 | 2.33 × 10\hat{ }2 | 9.3 × 10\hat{ }3 | |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%. The terms “between” and “at least” as used herein are inclusive. For example, a range of “between 5 and 10” means any amount equal to or greater than 5 but equal to or smaller than 10.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for detection of probiotic bacteria strains intentionally provided to animals comprising:
providing an animal with a known amount or number of probiotic bacteria;
following a pre-determined time, obtaining a biological sample suspected of comprising the inoculated probiotic bacteria from the animal; and
quantitatively detecting the amount of probiotic bacteria in the biological sample.

2. The method of claim 1, further comprising the step of modifying the amount of probiotic bacteria inoculated into a feed based on the amount wherein the probiotic bacteria detected in the biological sample.

3. The method of claim 1, wherein the step of quantitatively detecting the amount of probiotic bacteria in the biological sample is by a nucleic acid amplification assay, a nucleic acid detection assay, an oligonucleotide ligation assay (OLA), a primer probe assay, a polymerase chain reaction (PCR), a quantitative polymerase chain reaction (qPCR), multiplex-PCR, multiplex qPCR, a reverse-transcriptase polymerase chain reaction (RT-PCR), a ligase chain reaction (LCR), a polynomial amplification method, DNA sequencing method, or an method comprising primer extension.

4. The method of claim 1, wherein the step of quantitatively detecting the amount of probiotic bacteria in the biological sample is by qPCR it is selected from at least one of competitive, noncompetitive, or kinetic.

5. The method of claim 1, wherein the probiotic is a combination of a lactic acid producing bacterium and a lactic acid utilizing bacterium.

6. The method of claim 1, wherein the pre-determined amount of probiotic bacterium is selected from at least one of 1×10², 1×10³, 1×10⁴, 1×10⁵, 1×10⁶, 1×10⁷, 1×10⁸, 1×10⁹, or 1×10¹⁰ cfu per gram of a feed.

7. A method for quantifying a probiotic bacteria in a sample, said method comprising:
providing an animal with a feed inoculant comprising a known amount or number of probiotic bacteria;
following a pre-determined time, obtaining a biological sample suspected of comprising the inoculated probiotic bacteria from the animal;
generating a DNA fragment by amplifying a segment of genetic material of the probiotic bacterium; and
quantifying the amount of the probiotic bacterium, wherein the amplification using a pair of oligonucleotide primers, wherein the pair of oligonucleotides has at least 90% sequence identity with oligonucleotides of SEQ ID. No. 1 and SEQ ID. No. 2, respectively.

8. The method of claim 7, further comprising a step of culturing the probiotic bacterium in a medium and enumerating colonies formed by the lactic acid producing bacterium.

9. The method of claim 7, wherein the probiotic bacteria is selected from at least one of LA51 strain or PF24.

10. The method of claim 7, wherein the probiotic bacteria is mixed with animal feed.

11. The method of claim 7, wherein the biological sample is a fecal sample, a biopsy, a saliva sample, or a lymph node sample.

12. The method of claim 7, wherein the probiotic bacterium is selected from at least one of Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, Megasphaera elsdenii, Peptostreptococcus asaccharolyticus, Propionibacterium freudenreichii, Propionibacterium acidipropionici, Propionibacterium globosum, Propionibacterium jensenii, Propionibacterium shermanii, Propionibacterium spp., or Selenomonas ruminantium.

13. The method of claim 12, wherein the probiotic bacteria are a lactic acid producing bacterium is the LA51 strain, and the lactic acid utilizing bacterium is the PF24 strain.

14. A method for quantifying a lactic acid utilizing bacterium, said method comprising:
(a) providing an animal with a feed inoculant comprising a known amount or number of probiotic bacteria;
(b) generating a DNA fragment by amplifying a segment of genetic material of the lactic acid utilizing bacterium; and
(c) measuring the amount of the DNA fragment obtained in step (b) to quantify the amount of the lactic acid utilizing bacterium.

15. The method of claim 14, wherein the lactic acid utilizing bacterium is at least one of Lactobacillus strain NP51, Lactobacillus strain C28, Lactobacillus strain M35, Lactobacillus strain LA45, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, or Lactobacillus zeae.

16. A method for enumerating a lactic acid producing bacterium and a lactic acid utilizing bacterium in a sample, said method comprising:
providing an animal with a feed inoculant comprising a known amount or number of probiotic bacteria;
following a pre-determined time, obtaining a biological sample suspected of comprising the inoculated probiotic bacteria from the animal;
plating said sample or a portion thereof on a first medium to allow both lactic acid producing bacterium and lactic acid utilizing bacterium to grow;
plating said sample or a portion thereof on a second medium to allow only the lactic acid producing bacterium or only the lactic acid utilizing bacterium to grow on the second medium; and
counting the number of colonies on the first and second media to calculate the number of viable lactic acid producing bacteria and viable lactic acid utilizing bacteria in the sample.

17. The method of claim 16, wherein the lactic acid producing bacterium is the LA51 strain, and the lactic acid utilizing bacterium is the PF24 strain.

18. The method of claim 16, wherein only the lactic acid utilizing bacterium is capable of forming colonies on the second medium.

19. The method of claim 18, wherein the second medium comprises sodium lactate.

20. The method of claim 18, wherein the second medium comprises from 0.5% to 2% (w/vol) of sodium lactate.

21. The method of claim 18, wherein the second medium comprises 1% (w/vol) of sodium lactate.

22. The method of claim 16, further comprising the step of modifying the amount of probiotic bacteria inoculated into a feed based on the amount wherein the probiotic bacteria detected in the biological sample.

23. The method of claim 16, wherein the step of quantitatively detecting the amount of probiotic bacteria in the biological sample is by a nucleic acid amplification assay, a nucleic acid detection assay, an oligonucleotide ligation assay (OLA), a primer probe assay, a polymerase chain reaction (PCR), a quantitative polymerase chain reaction (qPCR), multiplex-PCR, multiplex qPCR, a reverse-transcriptase polymerase chain reaction (RT-PCR), a ligase chain reaction (LCR), a polynomial amplification method, DNA sequencing method, or an method comprising primer extension.

24. A composition comprising an oligonucleotide, wherein said oligonucleotide has at least 90% sequence identity with an oligonucleotide having a sequence selected from at least one of SEQ ID. No. 1 or SEQ ID. No. 2.

25. The composition of claim 23, wherein said oligonucleotide has at least 95% sequence identity with an oligonucleotide of SEQ ID. No. 1 or SEQ ID. No. 2.

26. The composition of claim 23, wherein said oligonucleotide has a sequence that is 100% identical with an oligonucleotide of SEQ ID. No. 1 and SEQ ID. No. 2.

27. A method for detection of probiotic bacteria strains intentionally provided to animals comprising:
providing an animal with a feed having a known amount or number of probiotic bacteria; and
following a pre-determined time, obtaining a biological sample suspected of comprising the inoculated probiotic bacteria from the animal; and
quantitatively detecting the amount of probiotic bacteria in the biological sample.

28. The method of claim 27, further comprising the step of modifying the amount of probiotic bacteria inoculated into the feed based on the amount wherein the probiotic bacteria detected in the biological sample.

29. The method of claim 27, wherein the step of quantitatively detecting the amount of probiotic bacteria in the biological sample is by a nucleic acid amplification assay, a nucleic acid detection assay, an oligonucleotide ligation assay (OLA), a primer probe assay, a polymerase chain reaction (PCR), a quantitative polymerase chain reaction (qPCR), multiplex-PCR, multiplex qPCR, a reverse-transcriptase polymerase chain reaction (RT-PCR), a ligase chain reaction (LCR), a polynomial amplification method, DNA sequencing method, or an method comprising primer extension.

30. The method of claim 27, wherein the step of quantitatively detecting the amount of probiotic bacteria in the biological sample is by qPCR it is selected from at least one of competitive, noncompetitive, or kinetic.

31. The method of claim 27, wherein the probiotic is a combination of a lactic acid producing bacterium and a lactic acid utilizing bacterium.

32. The method of claim 27, wherein the pre-determined amount of probiotic bacterium is selected from at least one of 1×10², 1×10³, 1×10⁴, 1×10⁵, 1×10⁶, 1×10⁷, 1×10⁸, 1×10⁹, or 1×10¹⁰ cfu per gram of a feed.

33. The method of claim 27, wherein the probiotic bacterium is selected from at least one of Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, Megasphaera elsdenii, Peptostreptococcus asaccharolyticus, Propionibacterium freudenreichii, Propionibacterium acidipropionici, Propionibacterium globosum, Propionibacterium jensenii, Propionibacterium shermanii, Propionibacterium spp., or Selenomonas ruminantium.
